(19) United States
(12) Patent Application Publication
(10) Pub. No.: US 2005/0020952 A1
(43) Pub. Date: Jan. 27, 2005

(54) Pad with aircell for an orthopedic brace

(76) Inventors: Erez Pick, Roslyn Heights, NJ (US); Fabian McCarthy JR., Basking Ridge, NJ (US)

Correspondence Address: ROPES & GRAY LLP, ONE INTERNATIONAL PLACE, BOSTON, MA, 02110-2624, US

(21) Appl. No.: 10/863,639

(22) Filed: Jun. 7, 2004

Related U.S. Application Data
(60) Provisional application No. 60/476,783, filed on Jun. 6, 2003.

(51) Int. Cl. A61F 5/00

(52) U.S. Cl. 602/27

(57) ABSTRACT

A pad for use with an orthopedic device comprises a sealed pre-inflated aircell disposed between a foam pad and the inner surface of a portion of the orthopedic device. The pads that may be employed with orthopedic devices such as braces and supports. In one embodiment, the pads may be removable and replaceable and fit within a brace, typically between the body part being braced and a support structure, such as a plastic shell support.

### CROSS-REFERENCE TO RELATED APPLICATIONS

This application claims priority to, and asserts every other benefit of, U.S. provisional patent application having Ser. No. 60/476783, filed on Jun. 6, 2003; the content of said U.S. provisional patent application is hereby incorporated by reference, in its entirety.

### BACKGROUND

This invention relates to a pad for use with an orthopedic brace. More particularly, this invention relates to a pad for use with an orthopedic brace wherein the pad includes a preinflated aircell. This invention also relates to an orthopedic brace comprising such a pad.

It is known in the orthopedic arts to provide braces for injured limbs, to provide support for the injured limb during the healing process. It is further known to provide pads or linings on the inner surfaces of such braces to promote comfort for the user and to encourage patient compliance with use of the brace. In some such orthopedic braces, the pad can include aircells to provide pneumatic pressure to the site of an injury and further promote the healing process. For example, U.S. Pat. No. 4,280,489, U.S. Pat. No. 4,628,945 and U.S. Pat. No. 5,125,400 each disclose ankle braces having aircells on the inner surfaces thereof to promote healing of an injured ankle; U.S. Pat. No. 4,872,448 discloses a knee brace having an inflatable aircell; and U.S. Pat. No. 5,577,998 discloses a walking brace having an aircell. Each of these patents is assigned to the instant assignee and is incorporated herein by reference in its entirety.

It is an object of the invention to provide a lining or pad for an orthopedic brace of any of the various types described above, which lining or pad is comfortable to the user.

It is another object of the invention to provide a lining or pad for an orthopedic brace of any of the various types described above, which lining or pad promotes the healing process for the injured limb.

It is still another object of the invention to provide a lining or pad for an orthopedic brace of any of the various types described above, which lining or pad is of simple construction.

It is yet another object of the invention to provide a lining or pad for an orthopedic brace of any of the various types described above, which lining or pad is relatively inexpensive.

### SUMMARY OF THE INVENTION

In accordance with the invention, a lining for use with an orthopedic device comprises a layer of foam disposed between layers of fabric, said layers of foam and layers of fabric preferably being substantially co-extensive with the inner surface of the orthopedic brace in contact with the patient, and further comprising a preinflated aircell fixedly secured to the surface of the pad not in contact with patient, said preinflated aircell being at a location so as to provide pneumatic pressure to the site of the injury. More particularly, the systems and methods described herein include a pad for providing orthopedic support, comprising a first fabric enclosure defining an interior cavity, a foam pad disposed within said interior cavity, a second fabric enclosure defining an interior cavity, an air cell being disposed within said interior cavity, and wherein said second fabric enclosure attaches to said first fabric enclosure. Optionally the fabric enclosures may comprises a fabric including Nylon. Further, in certain embodiments the air cell is dimensionally smaller than said foam pad, and the first fabric enclosure is positioned at a location selected to locate the air cell proximate to an edema. The air cell may comprise an air bladder, foam pad, a liquid bladder or some other type of bladder, and the air cell may attach to the exterior surface of the first fabric enclosure or be disposed within the first fabric enclosure. In other aspects, the invention provides methods for providing a pad for an orthopedic support.

### DESCRIPTION OF THE DRAWINGS

The foregoing and other objects and advantages of the invention will be appreciated more fully from the following further description thereof, with reference to the accompanying drawings wherein;

FIG. 1 illustrates a typical ankle brace of the prior art, fitted with an aircell of the invention.

FIG. 2 is a plan view of the inner surface of an ankle brace pad made in accordance with the invention.

FIG. 3 is a plan view of the outer surface of the ankle brace pad of FIG. 1.

FIG. 4 is a cross-sectional view of the ankle brace pad of FIGS. 2 and 3, with the thickness dimensions exaggerated for ease of understanding.

### DETAILED DESCRIPTION

The systems and methods described herein include pads that may be employed with orthopedic devices such as braces and supports. As will be described in more detail below, the pads of the invention may be, in one embodiment, removable and replaceable pads that can within a brace, typically between the body part being braced and a support structure, such as a plastic shell support. The pad may include an soft resilient support, such as an air cell that can provide pneumatic action for treating an edema at the site of injury. The air cell may be preinflated by having inserted therein a pad of material, for example compressible, resilient, porous material, such as open cell urethane foam, shaped and sized to substantially completely fill the interior volume of each airbag, respectively. However, it will be understood by those of skill in the art that the air cell may employ other filler materials, such as air, liquid, closed cell foam, or any other suitable material. The following detailed description of a preferred embodiment of the invention will be made with respect to an embodiment of the pad in the form of a pad for an ankle brace. It will be appreciated, however, that the invention is not so limited, and that pad of the instant invention can be configured for use with knee braces, wrist braces, back braces, walking braces, and other orthopedic braces. The size and shape of the pad may be altered according to the Application, and each brace may include one, or a plurality of pads, with the number selected as a function of the Application.

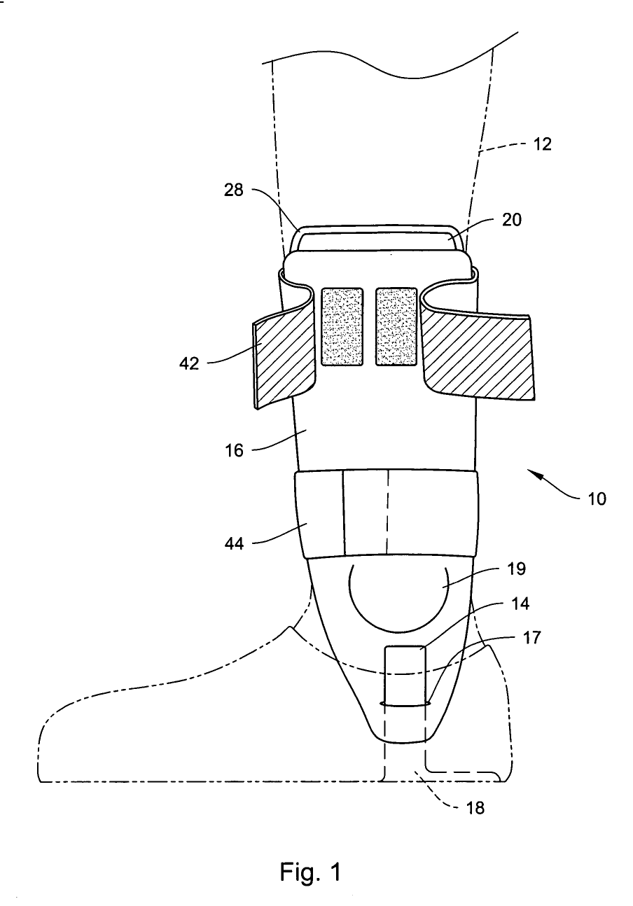

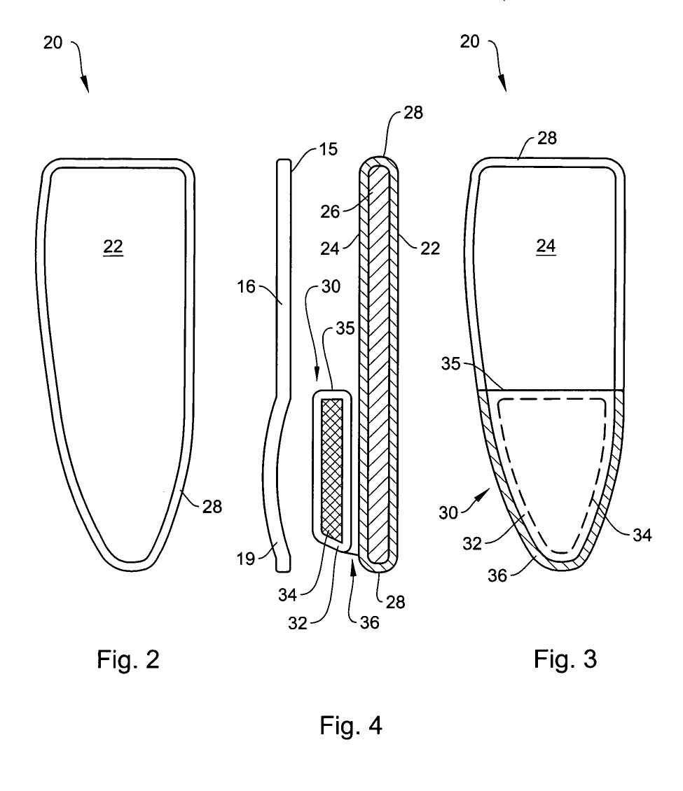

FIG. 1 illustrates a typical ankle brace of the prior art, such as generally illustrated and described in the aforementioned U.S. Pat. No. 4,280,489, but provided with an improved pad in accordance with the instant invention. For purposes of illustration, ankle brace 10 is shown in FIG. 1 fitted about a person's right lower leg and ankle, with the lower leg 12 being indicated in outline form by broken lines. In the illustrated embodiment, ankle brace 10 comprises a pair of opposed sidewall portions 16, only one of which is shown in the illustration. Each sidewall portion 16 is joined to a base portion 18 indicated in outline form by broken lines, such as by fitting a tab 19 of base portion 18 through a slot 17 in sidewall portion 16, the tab 14 of base portion 18 being affixed to an area of the outer surface of sidewall portion 16 such as by mating patches of hook-and-loop materials, as is known in the art. The brace is releasably secured to the user by means of flexible straps 42, 44, which can be secured to the sidewall portion outer surface by patches of hook-and-loop material. A pad 20 is disposed on the inwardly facing surface of each sidewall portion 16 in a generally juxtaposed manner substantially coextensive therewith. Each sidewall portion 16 extends longitudinally from base portion 18 and has an arcuate transverse cross-sectional shape sufficient to conform generally to the circumferential contour of the lower leg and ankle. In addition, the width of each sidewall portion gradually tapers from a minimum where it joins the base portion to a maximum at approximately the halfway point of its longitudinal extent. Each sidewall portion may have a curved area 19 in the region of the malleolus to accommodate the protrusion of the malleolus.

Pad 20 has an inwardly facing layer 22 that is juxtaposed to the limb of a user, and an outwardly facing layer 24 that is juxtaposed to the inner surface of sidewall portion 16, with a layer of foam 26 therebetween. Inwardly facing layer 22 and outwardly facing layer 26 are made of fabrics selected on the basis of their feel against the skin of the user, their ventilation properties, and their washability; they facilitate bonding to the plastic film used in the aircell, as described more fully below. Fabrics having a proportion of Nylon facilitate bonding to the plastic film and Nylon blends are generally suitable. One particularly suitable fabric in inwardly facing layer 22 is a mesh fabric made of a blend of 75% nylon and 25% polyester. Outwardly facing layer 24 is advantageously made of a fabric such as a 100% nylon V-loop fabric. Foam layer 26 can be either open cell foam or closed cell foam; open cell foam will be used where it is desired that the pad be permeable to air and/or moisture. The foam can be on the order of ⅜″ thick. A polyester/polyurethane foam is suitable for this purpose. The inwardly facing layer 22, the foam layer 26, and the outwardly facing layer 24 can be laminated together. The perimeter 28 of pad 20 is a closed seam.

Aircell 30 comprises a piece of plastic film 32 folded over a piece of foam 34, indicated in phantom lines in FIG. 3 and in cross section in FIG. 4. In particular, the film 32 of aircell 30 is folded at fold line 35 and sealed along its remaining open edges 36 to outwardly facing layer 24 of pad 20. Advantageously, aircell 30 can be created and sealed to layer 24 of pad 20 in a single step, such as by RF heat sealing, if the plastic film of aircell 30 is of a material that will bond to the fabric of layer 24. Plastic film 32 will be a strong, air-impermeable flexible material that can be operated on by standard heatsealing techniques. Polyvinyl chloride films and polyurethane films are suitable for this purpose; these films will adhere to a nylon-containing film under standard RF sealing parameters. The foam used can be either open-cell foam or closed-cell foam; one type of foam that is suitable is open-celled polyurethane foam. When the aircell 30 is sealed, an amount of air will be trapped inside; this trapped air will provide pneumatic compression to the site of an injury, as described below.

Aircell 30 is positioned on outwardly facing surface of pad 20 so as to be juxtaposed between foam layer 26 and the inner surface 15 of sidewall portion 16. Pad 20 is affixed to inner surface 15 of sidewall portion 16 by adhesives or by mating patches of hook-and-loop material. Aircell 30 will be positioned on pad 20 in a location that will provide pneumatic compression to an injury to promote healing. In the illustrated embodiment in which the pad 20 is to be used with an ankle brace, the aircell 30 is positioned to be received in the curved area 19 of sidewall portion 16 that accommodates the malleolus. The aircell 30 facilitates healing of the injury while providing greater comfort for the user. It will be appreciated that the pad 20 is of simple construction and is relatively inexpensive to manufacture.

The instant invention also encompasses orthopedic braces of various types having the pad as described and illustrated above. Those skilled in the art will know or be able to ascertain using no more than routine experimentation, many equivalents to the embodiments and practices described herein. For example, the systems have been described with reference to certain materials, such as Nylon and polyester. However, it will be apparent to those of skill in the art, that these materials are merely representative of the types of materials that may be employed with the systems described herein and that any suitable material may be employed. Accordingly, it will be understood that the invention is not to be limited to the embodiments disclosed herein, but is to be understood from the following claims, which are to be interpreted as broadly as allowed under the law.

1. A pad for providing orthopedic support, comprising
a first fabric enclosure defining an interior cavity,
a foam pad disposed within said interior cavity
a second fabric enclosure defining an interior cavity,
an air cell being disposed within said interior cavity, and
wherein said second fabric enclosure attaches to said first fabric enclosure.

2. The pad according to claim 1, wherein said first fabric enclosure comprises a fabric including Nylon.

3. The pad according to claim 1, wherein said second fabric enclosure comprises a fabric including Nylon.

4. The pad according to claim 1, wherein said air cell is dimensionally smaller than said foam pad,

5. The pad according to claim 4, wherein said air cell is attached to said first fabric enclosure at a location selected to locate the air cell proximate to an edema.

6. The pad according to claim 1, wherein the air cell comprises an air bladder.

7. The pad according to claim 1, wherein the air cell comprises an open cell foam pad.

8. The pad according to claim 1, further comprising an adhesive material disposed on a surface of the first fabric enclosure.

9. The pad according to claim 1, wherein the air cell is disposed within the first fabric enclosure.

10. The pad according to claim 1, wherein the air cell comprises a fluid filled bladder.

11. A method for providing a pad for an orthopedic support, comprising
providing a first fabric enclosure defining an interior cavity and inserting a
foam pad within said interior cavity,
providing a second fabric enclosure defining an interior cavity and inserting
an air cell being disposed within said interior cavity, and
attaching said second fabric enclosure to said first fabric enclosure.
